(19) United States
(12) Patent Application Publication
(10) Pub. No.: US 2025/0009622 A1
(43) Pub. Date: Jan. 9, 2025

(54) Silicon-free shampoo with good hair care efficacy

(71) Applicant: Pro Cosmetic Technology(Shanghai) Co., Ltd., Shanghai (CN)

(72) Inventors: Shuixing Chi, Shanghai (CN); Zifan Wu, Shanghai (CN); Junli Cai, Shanghai (CN); Yao Wang, Shanghai (CN)

(73) Assignee: Pro Cosmetic Technology(Shanghai) Co., Ltd., Shanghai (CN)

(21) Appl. No.: 18/762,370

(22) Filed: Jul. 2, 2024

(30) Foreign Application Priority Data
Jul. 3, 2023 (CN) 202310805717.2

(51) Int. Cl. A61K 8/37; A61Q 5/02; A61Q 5/12

(52) U.S. Cl. CPC A61K 8/375; A61Q 5/02; A61Q 5/12; A61K 2800/34; A61K 2800/805

(57) ABSTRACT

The present invention relates to the technical field of daily chemicals, in particular to a silicon-free shampoo with good hair care efficacy. The silicon-free shampoo with good hair care efficacy comprises the following components in percent by weight: 3% to 10% of a hair conditioner, 30% to 40% of an anionic surfactant, 2% to 4% of cocamidopropyl betaine, 4% to 8% of coconut oil monopropylene glycol amide, 0.8%-3.0% of a thickener, 0.10% to 2% of pH regulator, 0.4% to 1.0% of a preservative, 0.05% to 0.14% of a chelating agent, 10% to 18% of propylene glycol, and water for the surplus; the hair conditioner is formed by mixing water-soluble polyquaternium, hydrophobic modified titanium dioxide and glycerin. The silicon-free shampoo prepared by the present invention can effectively solve the problem of hair tangling during a hair washing process.

This application claims priority to Chinese Patent Application 202310805717.2, filed on Jul. 3, 2023, which is incorporated herein by reference.

### TECHNICAL FIELD

The present invention relates to the technical field of daily chemicals, in particular to a silicon-free shampoo with good hair care efficacy.

### BACKGROUND

Silicon oil is a key raw material widely applied to hair conditioning shampoos in recent decades. However, the non-degradability of silicon oil in the natural environment greatly limits the application of silicon oil to natural or environmentally friendly cosmetics. In the prior art, skin cream, waxes and cationic polymers are often used to replace silicon oils to prepare silicon-free shampoos.

Polyquaternium is the most commonly used cationic polymer to replace silicon oil. Polyquaternium can be adsorbed on the surfaces of negatively charged hairs to form a protective layer, which can reduce the friction resistance of hairs, repair damaged hair cuticles, and enhance the strength and flexibility of hair keratin. However, during the hair washing process, when polyquaternium is deposited on the surfaces of the negatively charged hairs, the polyquaternium can often absorb the surrounding negatively charged hairs, causing hair tangling, increasing the friction between hairs, and further resulting in hair damage during rubbing.

### SUMMARY

Therefore, the object of the present invention is to provide a silicon-free shampoo with good hair care efficacy, so as to solve the problem that existing silicon-free shampoos can cause hair tangling during a hair washing process.

Based on the aforementioned purpose, the present invention provides a silicon-free shampoo with good hair care efficacy, which includes the following components in percent by weight: 3% to 10% of a hair conditioner, 30% to 40% of an anionic surfactant, 2% to 4% of cocamidopropyl betaine, 4% to 8% of coconut oil monopropylene glycol amide, 0.8%-3% of a thickener, 0.1% to 2% of a pH regulator, 0.4% to 1% of a preservative, 0.05% to 0.14% of a chelating agent, 10% to 18% of propylene glycol, and water for the surplus.

The hair conditioner comprises the following components in percent by weight: 0.05%-to 5% of water-soluble polyquaternium, 5% to 10% of hydrophobic modified titanium dioxide, 0.01%-2.0% of lauroyl lysine, 0.01%-2.0% of glutamine, 0.01% to 2% of lysine PCA, 0.01% to 5% of trimethylolpropane trieste, 0.01% to 5% of hydrogenated polydecene, 0.01% to 5% of glycerin, 0.01% to 5% of argania spinosa oil, 0.01% to 5% of polyglycery-2 triisostearate, 1% to 10% of PEG-7 glyceryl cocoate, 0.01% to 2% of sodium PCA, 0.01% to 2% of sodium lactate, 0.01% to 2% of sodium citrate, 0.01% to 2% of glutamic acid, and water for the surplus.

The hydrophobic modified titanium dioxide is obtained by grafting titanium dioxide with octadecyl glycidyl ester.

Preferably, the anionic surfactant is one or more of lauramidopropyl hydroxysultaine, sodium methyl cocoyl taurate, sodium cocoyl alaninate, sodium lauroamphoacetate, sodium lauryl sulfate, sodium laureth sulfate, and sodium laureth sulfate.

Preferably, the water-soluble polyquaternium is one or more of polyquaternium-7, polyquaternium-49, guar hydroxypropyltrimonium chloride and polyquaternium-67.

Preferably, the thickener is one or more of sodium chloride, ammonium chloride, cocamide MEA, PEG-150 distearate and PEG-120 methyl glucose dioleate.

Preferably, the pH regulator is selected from one of citric acid and sodium citrate.

Preferably, the preservative is selected from one or more of antodyne, sodium benzoate, 1,2-hexanediol and p-hydroxyacetophenone.

Preferably, the chelating agent is one or more of sodium glucoheptonate, disodium edetate and tetrasodium EDTA.

Preferably, the particle size of the titanium dioxide is 20 to 50 nm.

Preferably, the hydrophobic modified titanium dioxide comprises the following specific synthesis steps:
S1: adding titanium dioxide and octadecyl glycidyl ester to an N,N-dimethylformamide solvent, performing ultrasonic dispersion in an ultrasonic device, adding a sulfuric acid solution with a concentration of 4 wt %-6 wt %, ensuring that reaction is performed for 8 to 12 h at 90° C. to 110° C. in a nitrogen condition, performing filtration washing and vacuum drying, to obtain hydrophobic modified titanium dioxide.

Preferably, in step S1, the mass ratio of the titanium dioxide, N,N-dimethylformamide solvent and octadecyl glycidyl ester is 5-8:50-80:0.3-0.5.

Further, the present invention further provides a preparation method of a silicon-free shampoo with good hair care efficacy, comprising the specific preparation process as follows:
S2: adding water-soluble polyquaternium, hydrophobic modified titanium dioxide, lauroyl lysine, glutamine, lysine PCA, trimethylolpropane trieste, hydrogenated polydecene, glycerin, argania spinosa oil, polyglycery-2 triisostearate, PEG-7 glyceryl cocoate, sodium PCA, sodium lactate, sodium citrate and glutamic acid to water, and stirring for 30 to 50 min, to obtain a hair conditioner;
S3: adding an anionic surfactant, cocamidopropyl betaine and coconut oil monopropylene glycol amide into mixed liquor of water and propylene glycol, stirring and dispersing, then adding a chelating agent, stirring and dispersing, and heating the mixed liquor to 70° C. to 80° C., to obtain a mixed liquor A;
S4: adding the hair conditioner, a thickener and a preservative to the mixed liquor A, stirring and dispersing, cooling the mixed liquor to room temperature, then adding pH regulator, and stirring evenly to form a microemulsion preparation, to obtain the silicon-free shampoo with good hair care efficacy.

The present invention has the following beneficial effects that:

The present invention adopts a compound of the anionic surfactant and an amphoteric surfactant to improve the decontamination and cleaning capacity of the shampoo, and ensures that the water-soluble polyquaternium, hydrophobic modified titanium dioxide, hydroxyethyl cellulose, and PEG-7 glyceryl cocoate are added to form a new silicon-free shampoo. In the silicon-free shampoo, the water-soluble polyquaternium is located in the water phase outside micelles composed of the surfactants, while the hydrophobic modified titanium dioxide is located in the surfactant micelles, so that stable dispersion is formed through skillful modification and matching.

When the silicon-free shampoo of the present invention is used, the silicon-free shampoo can be directly coated on hairs. Polyquaternium in water phase can be adsorbed to the surfaces of negatively charged hairs, and then the polyquaternium carries surfactant micelles wrapping hydrophobic modified titanium dioxide to be deposited on the surfaces of the hairs, reducing the bonding between the hairs. During the rubbing process, the modified titanium dioxide can convert the sliding friction between the hairs into rolling friction, and PEG-7 glyceryl cocoate and hydroxyethyl cellulose can improve the lubricity and reduce the problems of hair damage and hair tangling during friction through the conversion of frictional property and improvement of the lubricity.

During the rinsing stage of the silicon-free shampoo used in the present invention, surfactants can take away the hydrophobic modified titanium dioxide and the oil in hairs, forming a protective layer of polyquaternium on the surfaces of the hairs, which can repair damaged hair cuticles and hair enhance the strength and flexibility of hair keratin.

### DETAILED DESCRIPTION OF EMBODIMENTS

In order to make the objectives, technical solutions and advantages of the present invention clearer, the following further describes the present invention in detail with reference to specific embodiments.

### Embodiment 1

A preparation method of a silicon-free shampoo with good hair care efficacy, comprises the specific preparation process as follows:
S1: adding 5 g of titanium dioxide and 0.3 g of octadecyl glycidyl ester to 50 g of an N,N-dimethylformamide solvent, performing ultrasonic dispersion in an ultrasonic device, adding a sulfuric acid solution with a concentration of 4 wt %, ensuring that reaction is performed for 8 h at 90° C. in a nitrogen condition, performing filtration washing and vacuum drying, to obtain hydrophobic modified titanium dioxide.
S2: adding 0.05 g of polyquaternium-67, 5 g of hydrophobic modified titanium dioxide, 0.04 g of lauroyl lysine, 0.06 g of glutamine, 0.2 g of lysine PCA, 0.3 g of trimethylolpropane trieste, 2 g of hydrogenated polydecene, 1 g of glycerin, 0.01 g of argania spinosa oil, 0.01 g of polyglycery-2 triisostearate, 3 g of PEG-7 glyceryl cocoate, 1 g of sodium PCA, 0.1 g of sodium lactate, 0.5 g of sodium citrate and 1.2 g of glutamic acid to 85.03 g of water, and stirring for 30 min, to obtain a hair conditioner;
S3: adding 10 g of sodium cocoyl alaninate, 20 g of sodium methyl cocoyl taurate, 2 g of cocamidopropyl betaine, and 4 g of coconut oil monopropylene glycol amide into mixed liquor of 41.65 g of water and 18 g of propylene glycol, stirring and dispersing, then adding 0.05 g of disodium edetate, stirring and dispersing, and heating the mixed liquor to 70° C., to obtain a mixed liquor A;
S4: adding 3 g of the hair conditioner, 0.8 g of a thickener and 0.4 g of antodyne to the mixed liquor A, stirring and dispersing, cooling the mixed liquor to room temperature, then adding 0.1 g of citric acid, and stirring evenly to form a microemulsion preparation, to obtain the silicon-free shampoo with good hair care efficacy.

### Embodiment 2

A preparation method of a silicon-free shampoo with good hair care efficacy, comprises the specific preparation process as follows:
S1: adding 7 g of titanium dioxide and 0.4 g of octadecyl glycidyl ester to 65 g of an N,N-dimethylformamide solvent, performing ultrasonic dispersion in an ultrasonic device, adding a sulfuric acid solution with a concentration of 5 wt %, ensuring that reaction is performed for 10 h at 100° C. in a nitrogen condition, performing filtration washing and vacuum drying, to obtain hydrophobic modified titanium dioxide.
S2: adding 2 g of polyquaternium-7, 7 g of hydrophobic modified titanium dioxide, 1 g of lauroyl lysine, 1 g of glutamine, 1 g of lysine PCA, 1 g of trimethylolpropane trieste, 1 g of hydrogenated polydecene, 5 g of glycerin, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 67 g of water, and stirring for 50 min, to obtain a hair conditioner;
S3: adding 15 g of sodium cocoyl alaninate, 20 g of sodium methyl cocoyl taurate, 3 g of cocamidopropyl betaine, and 6 g of coconut oil monopropylene glycol amide into mixed liquor of 31.7 g of water and 14 g of propylene glycol, stirring and dispersing, then adding 0.1 g of disodium edetate, stirring and dispersing, and heating the mixed liquor to 75° C., to obtain a mixed liquor A;
S4: adding 7 g of the hair conditioner, 2 g of a thickener and 0.7 g of antodyne to the mixed liquor A, stirring and dispersing, cooling the mixed liquor to room temperature, then adding 0.5 g of citric acid, and stirring evenly to form a microemulsion preparation, to obtain the silicon-free shampoo with good hair care efficacy.

### Embodiment 3

A preparation method of a silicon-free shampoo with good hair care efficacy, comprises the specific preparation process as follows:
S1: adding 8 g of titanium dioxide and 0.5 g of octadecyl glycidyl ester to 80 g of an N,N-dimethylformamide solvent, performing ultrasonic dispersion in an ultrasonic device, adding a sulfuric acid solution with a concentration of 6 wt %, ensuring that reaction is performed for 12 h at 110° C. in a nitrogen condition, performing filtration washing and vacuum drying, to obtain hydrophobic modified titanium dioxide.
S2: adding 5 g of polyquaternium-49, 10 g of hydrophobic modified titanium dioxide, 0.01 g of lauroyl lysine, 0.01 g of glutamine, 2 g of lysine PCA, 5 g of trimethylolpropane trieste, 2 g of hydrogenated polydecene, 5 g of glycerin, 5 g of argania spinosa oil, 1 g of polyglycery-2 triisostearate, 10 g of PEG-7 glyceryl cocoate, 2 g of sodium PCA, 0.01 g of sodium lactate, 2 g of sodium citrate and 2 g of glutamic acid to 23.97 g of water, and stirring for 50 min, to obtain a hair conditioner;
S3: adding 20 g of sodium cocoyl alaninate, 20 g of sodium methyl cocoyl taurate, 4 g of cocamidopropyl betaine, and 8 g of coconut oil monopropylene glycol amide into mixed liquor of 24.16 g of water and 10 g of propylene glycol, stirring and dispersing, then adding 0.14 g of disodium edetate, stirring and dispersing, and heating the mixed liquor to 80° C., to obtain a mixed liquor A;
S4: adding 10 g of the hair conditioner, 3 g of a thickener and 1 g of antodyne to the mixed liquor A, stirring and dispersing, cooling the mixed liquor to room temperature, then adding 1.2 g of citric acid, and stirring evenly to form a microemulsion preparation, to obtain the silicon-free shampoo with good hair care efficacy.

### Comparative Example 1

A preparation method of a shampoo, comprises the specific preparation process as follows:
S1: adding 2 g of polyquaternium-7, 7 g of titanium dioxide, 1 g of lauroyl lysine, 1 g of glutamine, 1 g of lysine PCA, 1 g of trimethylolpropane trieste, 1 g of hydrogenated polydecene, 5 g of glycerin, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 67 g of water, and stirring for 50 min, to obtain a hair conditioner;
S2: same as S3 in Embodiment 2;
S3: same as S4 in Embodiment 2.

### Comparative Example 2

A preparation method of a shampoo, comprises the specific preparation process as follows:
S1: adding 2 g of polyquaternium-7.1 g of lauroyl lysine, 1 g of glutamine, 1 g of lysine PCA, 1 g of trimethylolpropane trieste, 1 g of hydrogenated polydecene, 5 g of glycerin, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 74 g of water, and stirring for 50 min, to obtain a hair conditioner;
S2: same as S3 in Embodiment 2;
S3: same as S4 in Embodiment 2.

### Comparative Example 3

A preparation method of a silicon-free shampoo, comprises the specific preparation process as follows:
S1: same as Embodiment 2;
S2: adding 7 g of hydrophobic modified titanium dioxide, 1 g of lauroyl lysine, 1 g of glutamine, 1 g of lysine PCA, 1 g of trimethylolpropane trieste, 1 g of hydrogenated polydecene, 5 g of glycerin, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 69 g of water, and stirring for 50 min, to obtain a hair conditioner;
S3: same as Embodiment 2;
S4: same as Embodiment 2.

### Comparative Example 4

A preparation method of a silicon-free shampoo, comprises the specific preparation process as follows:
S1: same as Embodiment 2;
S2: adding 2 g of polyquaternium-7, 7 g of hydrophobic modified titanium dioxide, 1 g of lauroyl lysine, 1 g of glutamine, 1 g of lysine PCA, 1 g of trimethylolpropane trieste, 1 g of hydrogenated polydecene, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 72 g of water, and stirring for 50 min, to obtain a hair conditioner;
S3: same as Embodiment 2;
S4: same as Embodiment 2.

### Comparative Example 5

A preparation method of a silicon-free shampoo, comprises the specific preparation process as follows:
S1: adding 2 g of polyquaternium-7.1 g of lauroyl lysine, 1 g of glutamine, 1 g of lysine PCA, 1 g of trimethylolpropane trieste, 1 g of hydrogenated polydecene, 3 g of argania spinosa oil, 2 g of polyglycery-2 triisostearate, 5 g of PEG-7 glyceryl cocoate, 1 g of sodium PCA, 1 g of sodium lactate, 1 g of sodium citrate and 1 g of glutamic acid to 79 g of water, and stirring for 50 min, to obtain a hair conditioner;
S2: same as S3 in Embodiment 2;
S3: same as S4 in Embodiment 2.

### Performance Test

Stability test: the silicon-free shampoos prepared in Embodiments 1 to 3 and Comparative Examples 1 to 5, and observe the statues after the shampoos are left to stand at room temperature for 30d and 60d. The results are as shown in Table 1.

Foam performance test: add 2.0 g of anhydrous calcium chloride and 1.48 g of anhydrous magnesium sulfate to 2000 mL of deionized water, and perform stirring and dissolving, to obtain hard water with a hardness of 1500 mg/kg. Add the 2.5 g of shampoos prepared in Embodiments 1 to 3 and Comparative Examples 1 to 5 to 100 mL of hard water with a hardness of 1500 mg/kg. Perform stirring to dissolve samples evenly. Dilute the shampoos to fix the volume of 1000 mL with deionized water, and heat the shampoos to 40° C. Absorb part of a test solution with a 200 mL quantitative funnel to rinse a foam meter along the wall of the foam meter. Then take and put the test solution to the bottom of the foam meter, and align the test solution with a standard scale to 50 mL. Absorb the test solution with a 200 mL quantitative funnel. Fix the center position of the funnel. Put down the test solution, and immediately write down the foam height, which is the initial height H₀ of the foam. Read the foam height H5 after 5 min. H₅/H₀ is a foam stabilizing coefficient. Repeatedly test each shampoo three times repeatedly and take an average value. The test results are as shown in Table 1.

TABLE 1
Stability and foam properties in Embodiments
1 to 3 and Comparative Examples 1 to 5

| | | | Initial | Foam | Foam |
|---|---|---|---|---|---|
| | | | height | height | stabilizing |
| | Stability | | H₀/cm of | H₅/cm after | coefficient |
| Sample | 30 d | 60 d | foam | 5 min | H₅/H₀ |
| Embodiment 1 | Uniform | Uniform | 18.32 | 17.44 | 0.952 |
| Embodiment 2 | Uniform | Uniform | 19.24 | 18.55 | 0.964 |
| Embodiment 3 | Uniform | Uniform | 18.36 | 17.11 | 0.932 |
| Comparative | Agglomerate | Agglomerate | 17.63 | 15.21 | 0.863 |
| Example 1 | | | | | |
| Comparative | Uniform | Uniform | 19.32 | 18.70 | 0.968 |
| Example 2 | | | | | |
| Comparative | Uniform | Uniform | 19.85 | 18.56 | 0.935 |
| Example 3 | | | | | |
| Comparative | Uniform | Uniform | 19.12 | 18.34 | 0.959 |
| Example 4 | | | | | |
| Comparative | Uniform | Uniform | 16.63 | 15.71 | 0.912 |
| Example 5 | | | | | |

Data analysis: it can be seen from Embodiments 1 to 3 that the silicon-free shampoo prepared by the present invention has long-lasting stability, excellent foaming property, and good foam stabilization. It can be seen from Embodiment 2 and Comparative Examples 1 and 2 that the addition of titanium dioxide without hydrophobic modification reduces the foaming property and foam stabilizing ability, and hydrophobic modification plays a crucial role in the stability of titanium dioxide in the shampoo of the present invention.

### Sensory Test

Select 20 volunteers and conduct a sensory test on the silicon-free shampoos of Embodiments 1 to 3 and Comparative Examples 1 to 5 in groups of three. The evaluation indexes are as shown in Table 2. The evaluation results are the average value of the scores of the 3 volunteers. The results are as shown in Table 3.

TABLE 2
Evaluation indexes of sensory test

| Item | Score | Feeling |
|---|---|---|
| Feeling | 1 | Hair tangling is severe; the resistance is very |
| during use | | strong; hairs are difficult to rinse. |
| | 2 | Hair tangling is relatively severe; the resistance is |
| | | relatively strong; hairs are relatively difficult to |
| | | rinse. |
| | 3 | Hair tangling is medium; the resistance is |
| | | medium; rising difficulty is medium. |
| | 4 | Hair tangling is slight; the resistance is relatively |
| | | weak; hairs are relatively easy to rinse. |
| | 5 | Hairs are not tangled; there is no resistance; hairs |
| | | are easy to rinse. |
| Feeling | 1 | Hair tangling is severe; the resistance is very |
| after use | | strong; hairs are difficult to comb. |
| | 2 | Hair tangling is relatively severe; the resistance is |
| | | relatively strong; hairs are relatively difficult to |
| | | comb. |
| | 3 | Hair tangling is medium; the resistance is |
| | | medium; combing difficulty is medium. |
| | 4 | Hair tangling is slight; the resistance is relatively |
| | | weak; hairs are relatively easy to comb. |
| | 5 | Hairs are not tangled; there is no resistance; hairs |
| | | are easy to comb. |

TABLE 3
Scores of sensory test in Embodiments
1 to 3 and Comparative Examples 1 to 5

| Sample | Feeling during use | Feeling after use |
|---|---|---|
| Embodiment 1 | 5 | 5 |
| Embodiment 2 | 5 | 5 |
| Embodiment 3 | 5 | 5 |
| Comparative Example 1 | 2 | 3 |
| Comparative Example 2 | 3 | 2 |
| Comparative Example 3 | 3 | 2 |
| Comparative Example 4 | 4 | 4 |
| Comparative Example 5 | 3 | 2 |

Data analysis: it can be seen from Embodiments 1 to 3 that the silicon-free shampoo with good hair care efficacy prepared by the present invention has no hair tangling and resistance, is easy to rinse during use, and after use, the status that hairs are not tangled, have no resistance, and are easy to comb can be maintained.

A person with ordinary skills in the art should understand that the discussion of any aforementioned embodiments is only illustrative, and is not intended to imply that the scope of the present invention (comprising the claims) is limited to these examples; under the spirit of the present invention, technical features in the aforementioned embodiments or different embodiments can also be combined, steps can be implemented in any order, and there are many other variations in different aspects of the present invention as described above, which are not provided in detail for the sake of simplification.

The present invention is intended to cover all such alternatives, modifications and variations falling within the broad scope of the appended claims. Therefore, any omission, modification, equivalent replacement and improvement made within the spirit and principle of the present invention shall be included within the protection scope of the present invention.

What is claimed is:

1. A silicon-free shampoo with good hair care efficacy, characterized by comprising the following components in percent by weight: 3% to 10% of a hair conditioner, 30% to 40% of an anionic surfactant, 2% to 4% of cocamidopropyl betaine, 4% to 8% of coconut oil monopropylene glycol amide, 0.8%-3% of a thickener, 0.1% to 2% of a pH regulator, 0.4% to 1% of a preservative, 0.05% to 0.14% of a chelating agent, 10% to 18% of propylene glycol, and water for the surplus;
the hair conditioner comprises the following components in percent by weight: 0.05%-to 5% of water-soluble polyquaternium, 5% to 10% of hydrophobic modified titanium dioxide, 0.01%-2.0% of lauroyl lysine, 0.01%-2.0% of glutamine, 0.01% to 2% of lysine PCA, 0.01% to 5% of trimethylolpropane trieste, 0.01% to 5% of hydrogenated polydecene, 0.01% to 5% of glycerin, 0.01% to 5% of argania spinosa oil, 0.01% to 5% of polyglycery-2 triisostearate, 1% to 10% of PEG-7 glyceryl cocoate, 0.01% to 2% of sodium PCA, 0.01% to 2% of sodium lactate, 0.01% to 2% of sodium citrate, 0.01% to 2% of glutamic acid, and water for the surplus;
the hydrophobic modified titanium dioxide is obtained by grafting titanium dioxide with octadecyl glycidyl ester;
the anionic surfactant is one or more of lauramidopropyl hydroxysultaine, sodium methyl cocoyl taurate, sodium cocoyl alaninate, sodium lauroamphoacetate, sodium lauryl sulfate, sodium laureth sulfate, and sodium laureth sulfate.

2. A silicon-free shampoo with good hair care efficacy according to claim 1, characterized in that the water-soluble polyquaternium is one or more of polyquaternium-7, polyquaternium-49, guar hydroxypropyltrimonium chloride and polyquaternium-67.

3. A silicon-free shampoo with good hair care efficacy according to claim 1, characterized in that the thickener is one or more of sodium chloride, ammonium chloride, cocamide MEA, PEG-150 distearate and PEG-120 methyl glucose dioleate.

4. A silicon-free shampoo with good hair care efficacy according to claim 1, characterized in that the pH regulator is selected from one of citric acid and sodium citrate.

5. A silicon-free shampoo with good hair care efficacy according to claim 1, characterized in that the preservative is selected from one or more of antodyne, sodium benzoate, 1,2-hexanediol and p-hydroxyacetophenone.

6. A silicon-free shampoo with good hair care efficacy according to claim 1, characterized in that the chelating agent is one or more of sodium glucoheptonate, disodium edetate and tetrasodium EDTA.

7. A silicon-free shampoo with good hair care efficacy according to claim 1, characterized in that the particle size of the titanium dioxide is 20 to 50 nm.

8. A silicon-free shampoo with good hair care efficacy according to claim 1, characterized in that the hydrophobic modified titanium dioxide comprises the following specific synthesis steps:
S1: adding titanium dioxide and octadecyl glycidyl ester to an N,N-dimethylformamide solvent, performing ultrasonic dispersion in an ultrasonic device, adding a sulfuric acid solution with a concentration of 4 wt %-6 wt %, ensuring that reaction is performed for 8 to 12 h at 90 to 110° C. in a nitrogen condition, performing filtration washing and vacuum drying, to obtain hydrophobic modified titanium dioxide.

9. A silicon-free shampoo with good hair care efficacy according to claim 8, characterized in that in step S1, the mass ratio of the titanium dioxide, N,N-dimethylformamide solvent and octadecyl glycidyl ester is 5-8:50-80:0.3-0.5.

10. A preparation method of a silicon-free shampoo with good hair care efficacy according to any one of claims 1 to 9, comprising the specific preparation process as follows:
S2: adding water-soluble polyquaternium, hydrophobic modified titanium dioxide, lauroyl lysine, glutamine, lysine PCA, trimethylolpropane trieste, hydrogenated polydecene, glycerin, argania spinosa oil, polyglycery-2 triisostearate, PEG-7 glyceryl cocoate, sodium PCA, sodium lactate, sodium citrate and glutamic acid to water, and stirring for 30 to 50 min, to obtain a hair conditioner;
S3: adding an anionic surfactant, cocamidopropyl betaine and coconut oil monopropylene glycol amide into mixed liquor of water and propylene glycol, stirring and dispersing, then adding a chelating agent, stirring and dispersing, and heating the mixed liquor to 70 to 80° C., to obtain a mixed liquor A;
S4: adding the hair conditioner, a thickener and a preservative to the mixed liquor A, stirring and dispersing, cooling the mixture liquor to room temperature, then adding pH regulator, and stirring evenly to form a microemulsion preparation, to obtain the silicon-free shampoo with good hair care efficacy.
